(19) United States
(12) Patent Application Publication
(10) Pub. No.: US 2020/0354756 A1
(43) Pub. Date: Nov. 12, 2020

(54) DISRUPTION OF MVB12 IN YEAST IS ASSOCIATED WITH INCREASED ALCOHOL PRODUCTION AND TOLERANCE

(71) Applicant: DANISCO US INC, Palo Alto, CA (US)

(72) Inventors: ZHONGQIANG CHEN, WILMINGTON, DE (US); STEVEN ADAM KANE, WILMINGTON, DE (US); CELIA EMILY GABY PAYEN, WILMINGTON, DE (US); GERDA SAXER QUANCE, KENNETT SQUARE, PA (US); LUAN TAO, WALLINGFORD, PA (US)

(21) Appl. No.: 16/839,558

(22) Filed: Apr. 3, 2020

Related U.S. Application Data
(60) Provisional application No. 62/828,772, filed on Apr. 3, 2019.

(51) Int. Cl. C12P 7/16; C12N 1/16

(52) U.S. Cl. CPC C12P 7/16; C12R 1/865; C12N 1/16

(57) ABSTRACT

Described are compositions and methods relating to yeast having a genetic mutation that results in increased alcohol production and tolerance, including increased ethanol production and increased butanol tolerance. Such yeast is well-suited for use in alcohol and butanol production to increase yields.

This application is related to and claims the benefit of priority of U.S. Provisional Application Ser. No. 62/828,772 filed on Apr. 3, 2019, the entirety of which is herein incorporated herein by reference.

The Sequence Listing associated with this application is filed in electronic form via EFS-Web and hereby incorporated by reference into the specification in its entirety.

### FIELD OF THE INVENTION

The present strains and methods relate to yeast having a genetic mutation that results in increased alcohol production and tolerance, including increased ethanol production and increased butanol tolerance. Such yeast is well-suited for use in alcohol and butanol production to increase yields.

### BACKGROUND

Many countries make fuel alcohol from fermentable substrates, such as corn starch, sugar cane, cassava, and molasses. According to the Renewable Fuel Association (Washington D.C., United States), fuel ethanol production in 2015 was close to 15 billion gallons in the United States, alone.

Butanol is an important industrial chemical and drop-in fuel component with a variety of applications including use as a renewable fuel additive, a feedstock chemical in the plastics industry, and a food-grade extractant in the food and flavor industry. Accordingly, there is a high demand for alcohols such as butanol and isobutanol, as well as for efficient and environmentally-friendly production methods.

In view of the large amount of alcohol produced in the world, even a minor increase in the efficiency of a fermenting organism can result in a significant increase in the amount of available alcohol. Accordingly, the need exists for organisms that are more efficient at producing alcohol.

### SUMMARY

Described are compositions and methods relating to yeast cells having a modified phenotype with respect to alcohol production and tolerance.

In one aspect, modified yeast cells derived from parental yeast cells are provided, the modified cells comprising a genetic alteration that causes the modified cells to produce a decreased amount of functional MVB12 polypeptides compared to the parental cells, wherein the modified cells have increased alcohol tolerance and/or decreased alcohol tolerance compared to the parental cells under equivalent fermentation conditions.

In some embodiments of the modified cells, the genetic alteration comprises a disruption of a YGR206W gene present in the parental cells.

In some embodiments of the modified cells, disruption of a YGR206W gene is the result of deletion of all or part of a YGR206W gene.

In some embodiments of the modified cells, disruption of a YGR206W gene is the result of deletion of a portion of genomic DNA comprising a YGR206W gene.

In some embodiments of the modified cells, disruption of a YGR206W gene is the result of mutagenesis of a YGR206W gene.

In some embodiments of the modified cells, disruption of a YGR206W gene is performed in combination with introducing a gene of interest at the genetic locus of a YGR206W gene.

In some embodiments of the modified cells, the cells do not produce functional MVB12 polypeptides.

In some embodiments of the modified cells, the cells do not produce MVB12 polypeptides.

In some embodiments of the modified cells, the cells further comprise an exogenous gene encoding a carbohydrate processing enzyme.

In some embodiments, the modified cells further comprise an alteration in the glycerol pathway and/or the acetyl-CoA pathway.

In some embodiments, the modified cells further comprise an alternative pathway for making alcohol.

In some embodiments of the modified cells, the cells are of a Saccharomyces spp.

In some embodiments of the modified cells, the cells produce an increased amount of ethanol compared to the parental cells.

In some embodiments of the modified cells, the cells produce a decreased amount of acetate compared to the parental cells.

In some embodiments of the modified cells, the cells have a reduced lag phase in the presence of butanol compared to the parental cells.

In some embodiments of the modified cells, the cells comprise an isobutanol biosynthetic pathway.

In some embodiments of the modified cells, the isobutanol biosynthetic pathway comprises a polynucleotide encoding a polypeptide that catalyzes a substrate to product conversion selected from the group consisting of: (a) pyruvate to acetolactate; (b) acetolactate to 2,3-dihydroxyisovalerate; (c) 2,3-dihydroxyisovalerate to α-ketoisovalerate; (d) α-ketoisovalerate to isobutyraldehyde; and (e) isobutyraldehyde to isobutanol.

In some embodiments, the modified cells comprise a genetic alteration that causes the modified cells to produce a decreased amount of functional MVB12 polypeptides compared to the parental cells and an isobutanol biosynthetic pathway.

In some embodiments, the modified cells further comprise reduced or eliminated pyruvate decarboxylase expression or activity. In some embodiments, the modified cells have reduced or eliminated PDC1, PDC5, or PDC6 activity or a combination thereof.

In some embodiments, the modified cells further comprise reduced or eliminated glycerol-3-phosphate dehydrogenase expression or activity. In some embodiments, the modified cells have reduced GPD2 activity.

In some embodiments, the modified cells further comprise reduced or eliminated FRA2 expression or activity.

In some embodiments, the modified cells comprise a genetic alteration that causes the modified cells to produce a decreased amount of functional MVB12 polypeptides compared to the parental cells, an isobutanol biosynthetic pathway, reduced or eliminated pyruvate decarboxylase expression or activity, reduced or eliminated glycerol-3-phosphate dehydrogenase expression or activity, and reduced or eliminated FRA2 expression or activity.

In some embodiments, the modified cells comprise a genetic alteration comprising a disruption of a YGR206W gene present in the parental cells and an isobutanol biosynthetic pathway. In some embodiments, the modified cells further comprise reduced or eliminated pyruvate decarboxylase expression or activity, reduced or eliminated glycerol-3-phosphate dehydrogenase expression or activity, reduced or eliminated FRA2 expression or activity, or combinations thereof.

In another aspect, a method for producing a modified yeast cell is provided, comprising: introducing a genetic alteration into a parental yeast cell, which genetic alteration reduces or prevents the production of functional MVB12 polypeptide compared to the parental cells, thereby producing modified cells that have during fermentation an increased alcohol tolerance and/or decreased alcohol tolerance compared to the parental cells under equivalent fermentation.

In some embodiments of the method, the genetic alteration comprises disrupting a YGR206W gene in the parental cells by genetic manipulation.

In some embodiments of the method, the genetic alteration comprises deleting a YGR206W gene in the parental cells using genetic manipulation.

In some embodiments of the method, disruption of a YGR206W gene is performed in combination with introducing a gene of interest at the genetic locus of a YGR206W gene.

In some embodiments of the method, disruption of a YGR206W gene is performed in combination with making an alteration in the glycerol pathway and/or the acetyl-CoA pathway.

In some embodiments of the method, disruption of a YGR206W gene is performed in combination with adding an alternative pathway for making alcohol.

In some embodiments of the method, disruption of a YGR206W gene is performed in combination with introducing an exogenous gene encoding a carbohydrate processing enzyme.

In some embodiments of the method, the modified yeast cell further comprises an isobutanol biosynthetic pathway.

In some embodiments of the method, the isobutanol biosynthetic pathway comprises a polynucleotide encoding a polypeptide that catalyzes a substrate to product conversion selected from the group consisting of: (a) pyruvate to acetolactate; (b) acetolactate to 2,3-dihydroxyisovalerate; (c) 2,3-dihydroxyisovalerate to α-ketoisovalerate; (d) α-ketoisovalerate to isobutyraldehyde; and (e) isobutyraldehyde to isobutanol.

In some embodiments of the method, the modified cell is from a Saccharomyces spp.

These and other aspects and embodiments of present modified cells and methods will be apparent from the description, including the accompanying Figures.

### DESCRIPTION OF THE DRAWINGS

FIG. 1 is a graph showing reduced acetate production by yeast harboring the deletion of the YGR206W gene, encoding the MVB12 polypeptide, after 55 hr incubation in the presence of liquefact.

FIG. 2 is a graph showing a reduced lag phase by yeast harboring the deletion of the YGR206W gene, encoding the MVB12 polypeptide, in media containing 5 g/L isobutanol.

### DESCRIPTION

Described are methods relating to yeast cells having a modified phenotype with respect to alcohol production and tolerance. When used for fuel alcohol production, the modified cells allow for increased yields and or shorter fermentation times, thereby increasing the supply of alcohol for world consumption.

### Definitions

Prior to describing the present strains and methods in detail, the following terms are defined for clarity. Terms not defined should be accorded their ordinary meanings as used in the relevant art.

As used herein, “alcohol” refers to an organic compound in which a hydroxyl functional group (—OH) is bound to a saturated carbon atom.

As used herein, “butanol” refers to the butanol isomers 1-butanol, 2-butanol, tert-butanol, and/or isobutanol (also known as 2-methyl-1-propanol) either individually or as mixtures thereof.

As used herein, “yeast cells,” “yeast strains,” or simply “yeast” refer to organisms from the phyla Ascomycota and Basidiomycota. Exemplary yeast is budding yeast from the order Saccharomycetales. Examples of yeast are Saccharomyces spp., including but not limited to Saccharomyces cerevisiae. Yeast include organisms used for the production of fuel alcohol as well as organisms used for the production of potable alcohol, including specialty and proprietary yeast strains used to make distinctive-tasting beers, wines, and other fermented beverages.

As used herein, the phrase “variant yeast cells,” “modified yeast cells,” or similar phrases (see above), refer to yeast that include genetic modifications and characteristics described herein. Variant/modified yeast do not include naturally occurring yeast.

The term “isobutanol biosynthetic pathway” refers to an enzyme pathway to produce isobutanol from pyruvate.

The term “acetolactate synthase” refers to an enzyme that catalyzes the conversion of pyruvate to acetolactate and CO₂. Examples of acetolactate synthases are known by the Enzyme Commission (EC) Number 2.2.1.6 (Enzyme Nomenclature 1992, Academic Press, San Diego). These enzymes are available from a number of sources, including, but not limited to, Bacillus subtilis [GenBank Nos: CAB15618 and Z99122, NCBI (National Center for Biotechnology Information) amino acid sequence, NCBI nucleotide sequence, respectively], Klebsiella pneumoniae (GenBank Nos: AAA25079 and M73842), and Lactococcus lactis (GenBank Nos: AAA25161 and L16975).

The term “ketol-acid reductoisomerase” (KARI) refers to an enzyme that catalyzes the conversion of acetolactate to 2,3-dihydroxyisovalerate. Suitable enzymes utilize NADH (reduced nicotinamide adenine dinucleotide) and/or NADPH as electron donor. Examples of ketol-acid reductoisomerases are known by the EC Number 1.1.1.86 and sequences are available from a vast array of microorganisms, including, but not limited to, Escherichia coli (GenBank Nos: NP_418222 and NC_000913), Saccharomyces cerevisiae (GenBank Nos: NP_013459 and NC_001144), Methanococcus maripaludis (GenBank Nos: CAF30210 and BX957220), and Bacillus subtilis (GenBank Nos: CAB14789 and Z99118). Examples of KARIs also include those from Lactococcus lactis, Vibrio cholera, Pseudomonas aeruginosa PAO1, Pseudomonas fluorescens PF5 and variants thereof. KARIs include Anaerostipes caccae KARI as well as variants are described in U.S. Pat. Nos. 10,174,345; 9,512,408; 9,422,581; 9,422,582; and 9,790,521, the entire contents of each are herein incorporated by reference.

The term “dihydroxy acid dehydratase” (DHAD) refers to an enzyme that catalyzes the conversion of 2,3-dihydroxyisovalerate to α-ketoisovalerate. Examples of dihydroxy acid dehydratases are known by the EC Number 4.2.1.9. These enzymes are available from a vast array of microorganisms, including, but not limited to, E. coli (GenBank Nos: YP_026248 and NC_000913), S. cerevisiae (GenBank Nos: NP_012550 and NC_001142), M maripaludis (GenBank Nos: CAF29874 and BX957219), and B. subtilis (GenBank Nos: CAB14105 and Z99115). Examples of dihydroxy acid dehydratase also include DHAD from Streptococcus mutans.

The term “branched-chain α-keto acid decarboxylase” refers to an enzyme that catalyzes the conversion of α-ketoisovalerate to isobutyraldehyde and CO₂. Examples of branched-chain α-keto acid decarboxylases are known by the EC Number 4.1.1.72 and are available from a number of sources, including, but not limited to, Lactococcus lactis (GenBank Nos: AAS49166, AY548760, CAG34226, and AJ746364), Salmonella typhimurium (GenBank Nos: NP_461346 and NC_003197), and Clostridium acetobutylicum (GenBank Nos: NP_149189 and NC_001988). Examples of branched-chain α-keto acid decarboxylases also include Listeria grayi, Lactococcus lactis, and Macrococcus caseolyticus as described in U.S. Pat. No. 9,169,467, the entire contents of which are herein incorporated by reference.

The term “alcohol dehydrogenase” (ADH) refers to an enzyme that catalyzes the conversion of isobutyraldehyde to isobutanol. Examples of alcohol dehydrogenases are known by the EC Number 1.1.1.265, but may also be classified under other alcohol dehydrogenases (specifically, EC 1.1.1.1 or 1.1.1.2). These enzymes utilize NADH (reduced nicotinamide adenine dinucleotide) and/or NADPH as electron donor and are available from a number of sources, including, but not limited to, S. cerevisiae (GenBank Nos: NP_010656, NC_001136; NP_014051; and NC_001145), E. coli (GenBank Nos: NP_417484 and NC_000913) and C. acetobutylicum (GenBank Nos: NP_349892, NC_003030; NP_349891, and NC_003030). Alcohol dehydrogenases also include horse liver ADH, Beijerinkia indica ADH, and ADH from Achromobacter xylosoxidans.

As used herein, the phrase “substantially free of an activity,” or similar phrases, means that a specified activity is either undetectable in an admixture or present in an amount that would not interfere with the intended purpose of the admixture.

As used herein, the terms “polypeptide” and “protein” (and their respective plural forms) are used interchangeably to refer to polymers of any length comprising amino acid residues linked by peptide bonds. The conventional one-letter or three-letter codes for amino acid residues are used herein and all sequence are presented from an N-terminal to C-terminal direction. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

As used herein, functionally and/or structurally similar proteins are considered to be “related proteins.” Such proteins can be derived from organisms of different genera and/or species, or even different classes of organisms (e.g., bacteria and fungi). Related proteins also encompass homologs determined by primary sequence analysis, determined by secondary or tertiary structure analysis, or determined by immunological cross-reactivity.

As used herein, the term “homologous protein” refers to a protein that has similar activity and/or structure to a reference protein. It is not intended that homologs necessarily be evolutionarily related. Thus, it is intended that the term encompass the same, similar, or corresponding enzyme(s) (i.e., in terms of structure and function) obtained from different organisms. In some embodiments, it is desirable to identify a homolog that has a quaternary, tertiary and/or primary structure similar to the reference protein. In some embodiments, homologous proteins induce similar immunological response(s) as a reference protein. In some embodiments, homologous proteins are engineered to produce enzymes with desired activity(ies).

The degree of homology between sequences can be determined using any suitable method known in the art (see, e.g., Smith and Waterman, Adv. Appl. Math. 2:482, 1981; Needleman and Wunsch, J. Mol. Biol. 48:443, 1970; Pearson and Lipman, Proc. Nat. Acad. Sci. USA 85:2444, 1988; programs such as GAP, BESTFIT, FASTA, and TFASTA in the Wisconsin Genetics Software Package (Genetics Computer Group, Madison, Wis.); and Devereux, et al., Nucleic Acids Res. 12:387-95, 1984).

For example, PILEUP is a useful program to determine sequence homology levels. PILEUP creates a multiple sequence alignment from a group of related sequences using progressive, pair-wise alignments. It can also plot a tree showing the clustering relationships used to create the alignment. PILEUP uses a simplification of the progressive alignment method of Feng and Doolittle, (Feng and Doolittle, J. Mol. Evol. 35:351-60, 1987). The method is similar to that described by Higgins and Sharp (CABIOS 5:151-53, 1989). Useful PILEUP parameters including a default gap weight of 3.00, a default gap length weight of 0.10, and weighted end gaps. Another example of a useful algorithm is the BLAST algorithm, described by Altschul, et al., (J. Mol. Biol. 215:403-10, 1990) and Karlin, et al. (Proc. Nat. Acad. Sci. USA 90:5873-87, 1993). One particularly useful BLAST program is the WU-BLAST-2 program (see, e.g., Altschul, et al., Meth. Enzymol. 266:460-80, 1996). Parameters “W,” “T,” and “X” determine the sensitivity and speed of the alignment. The BLAST program uses as defaults a word-length (W) of 11, the BLOSUM62 scoring matrix (see, e.g., Henikoff and Henikoff, Proc. Nat. Acad. Sci. USA 89:10915, 1989) alignments (B) of 50, expectation (E) of 10, M'S, N′-4, and a comparison of both strands.

As used herein, the phrases “substantially similar” and “substantially identical,” in the context of at least two nucleic acids or polypeptides, typically means that a polynucleotide or polypeptide comprises a sequence that has at least about 70% identity, at least about 75% identity, at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, or even at least about 99% identity, or more, compared to the reference (i.e., wild-type) sequence. Percent sequence identity is calculated using CLUSTAL W algorithm with default parameters. See Thompson et al., Nucleic Acids Res. 22:4673-4680, 1994. Default parameters for the CLUSTAL W algorithm are:
Gap opening penalty: 10.0
Gap extension penalty: 0.05
Protein weight matrix: BLOSUM series
DNA weight matrix: IUB
Delay divergent sequences %: 40
Gap separation distance: 8
DNA transitions weight: 0.50
List hydrophilic residues: GPSNDQEKR
Use negative matrix: OFF
Toggle Residue specific penalties: ON
Toggle hydrophilic penalties: ON
Toggle end gap separation penalty OFF.

Another indication that two polypeptides are substantially identical is that the first polypeptide is immunologically cross-reactive with the second polypeptide. Typically, polypeptides that differ by conservative amino acid substitutions are immunologically cross-reactive. Thus, a polypeptide is substantially identical to a second polypeptide, for example, where the two peptides differ only by a conservative substitution. Another indication that two nucleic acid sequences are substantially identical is that the two molecules hybridize to each other under stringent conditions (e.g., within a range of medium to high stringency).

As used herein, the term “gene” is synonymous with the term “allele” in referring to a nucleic acid that encodes and directs the expression of a protein or RNA. Vegetative forms of filamentous fungi are generally haploid, therefore a single copy of a specified gene (i.e., a single allele) is sufficient to confer a specified phenotype.

As used herein, the terms “wild-type” and “native” are used interchangeably and refer to genes proteins or strains found in nature.

As used herein, the term “protein of interest” refers to a polypeptide that is desired to be expressed in modified yeast. Such a protein can be an enzyme, a substrate-binding protein, a surface-active protein, a structural protein, a selectable marker, or the like, and can be expressed at high levels. The protein of interest is encoded by a modified endogenous gene or a heterologous gene (i.e., gene of interest) relative to the parental strain. The protein of interest can be expressed intracellularly or as a secreted protein.

As used herein, “deletion of a gene,” refers to its removal from the genome of a host cell. Where a gene includes control elements (e.g., enhancer elements) that are not located immediately adjacent to the coding sequence of a gene, deletion of a gene refers to the deletion of the coding sequence, and optionally adjacent enhancer elements, including but not limited to, for example, promoter and/or terminator sequences, but does not require the deletion of non-adjacent control elements.

As used herein, “disruption of a gene” refers broadly to any genetic or chemical manipulation, i.e., mutation, that substantially prevents a cell from producing a function gene product, e.g., a protein, in a host cell. Exemplary methods of disruption include complete or partial deletion of any portion of a gene, including a polypeptide-coding sequence, a promoter, an enhancer, or another regulatory element, or mutagenesis of the same, where mutagenesis encompasses substitutions, insertions, deletions, inversions, and combinations and variations, thereof, any of which mutations substantially prevent the production of a function gene product. A gene can also be disrupted using RNAi, antisense, or any other method that abolishes gene expression. A gene can be disrupted by deletion or genetic manipulation of non-adjacent control elements.

As used herein, the terms “genetic manipulation” and “genetic alteration” are used interchangeably and refer to the alteration/change of a nucleic acid sequence. The alteration can include but is not limited to a substitution, deletion, insertion or chemical modification of at least one nucleic acid in the nucleic acid sequence.

As used herein, a “primarily genetic determinant” refers to a gene, or genetic manipulation thereof, that is necessary and sufficient to confer a specified phenotype in the absence of other genes, or genetic manipulations, thereof. However, that a particular gene is necessary and sufficient to confer a specified phenotype does not exclude the possibility that additional effects to the phenotype can be achieved by further genetic manipulations.

As used herein, a “functional polypeptide/protein” is a protein that possesses an activity, such as an enzymatic activity, a binding activity, a surface-active property, or the like, and which has not been mutagenized, truncated, or otherwise modified to abolish or reduce that activity. Functional polypeptides can be thermostable or thermolabile, as specified.

As used herein, “a functional gene” is a gene capable of being used by cellular components to produce an active gene product, typically a protein. Functional genes are the antithesis of disrupted genes, which are modified such that they cannot be used by cellular components to produce an active gene product, or have a reduced ability to be used by cellular components to produce an active gene product.

As used herein, yeast cells have been “modified to prevent the production of a specified protein” if they have been genetically or chemically altered to prevent the production of a functional protein/polypeptide that exhibits an activity characteristic of the wild-type protein. Such modifications include, but are not limited to, deletion or disruption of the gene encoding the protein (as described, herein), modification of the gene such that the encoded polypeptide lacks the aforementioned activity, modification of the gene to affect post-translational processing or stability, and combinations, thereof.

As used herein, “attenuation of a pathway” or “attenuation of the flux through a pathway” i.e., a biochemical pathway, refers broadly to any genetic or chemical manipulation that reduces or completely stops the flux of biochemical substrates or intermediates through a metabolic pathway. Attenuation of a pathway may be achieved by a variety of well-known methods. Such methods include but are not limited to: complete or partial deletion of one or more genes, replacing wild-type alleles of these genes with mutant forms encoding enzymes with reduced catalytic activity or increased Km values, modifying the promoters or other regulatory elements that control the expression of one or more genes, engineering the enzymes or the mRNA encoding these enzymes for a decreased stability, misdirecting enzymes to cellular compartments where they are less likely to interact with substrate and intermediates, the use of interfering RNA, and the like.

As used herein, “aerobic fermentation” refers to growth in the presence of oxygen.

As used herein, “anaerobic fermentation” refers to growth in the absence of oxygen.

As used herein, the singular articles “a,” “an,” and “the” encompass the plural referents unless the context clearly dictates otherwise. All references cited herein are hereby incorporated by reference in their entirety.

The following abbreviations/acronyms have the following meanings unless otherwise specified:
° C. degrees Centigrade
DNA deoxyribonucleic acid
DP degree of polymerization
ds or DS dry solids
g or gm gram
g/L grams per liter
GA glucoamylase
GAU/g ds glucoamylase units per gram dry solids
HPLC high performance liquid chromatography
hr or h hour
kDa kilodalton
M molar
mg milligram
mL or ml milliliter
ml/min milliliter per minute
mM millimolar
MVB multivesicular bodies
N normal
PCR polymerase chain reaction
ppm parts per million
SAPU/g ds protease units per gram dry solids
SSCU/g ds fungal α-amylase units per gram dry solids
Δ relating to a deletion
μg microgram
μL and μl microliter
μM and μm micromolar

### Modified Yeast Cells Expressing Reduced Levels of MVB12 Polypeptides

MVB12 is a 101-amino acid residue, 12-kDa protein that has the amino acid sequence of SEQ ID NO: 1:

MNNNVEELLR RIPLYNKYGK DFPQETVTRF QMPEFKLPAL
QPTRDLLCPW YEECDNITKV CQLHDSSNKK FDQWYKEQYL
SKKPPGIVGN TLLSPSRKDN S*

MVB12 is a component of multivesicular bodies (MVB), which are late endosome-containing internal vesicles formed following the inward budding of the outer endosomal membrane in yeast. The contents of MVB are released into the lysosome lumen and proteins present in the membrane of MVB are ultimately recycled by way of other compartments. MVB12 is a subunit of the cytoplasmatic endosomal sorting complex required for transport (ESCRT-I) necessary to stabilize core complex oligomers. The ESCRT-I complex is involved in ubiquitin-dependent sorting of proteins in the endosome. MVB12 appears to stabilize the ESCRT-I core proteins and negatively affects the interaction between ESCRT-I and ESCRT-II, thereby promoting MVB sorting (see, e.g., Chu, et al., J. Cell. Biol. 175:815-23, 2006; Oestreich, et al., Mol. Biol. Cell 18:646-57, 2007; Gill, et al., EMBO J. 26:600-12, 2007; Brookhart Shields, et al., J. Cell Biol. 185:213-24, 2009; and Peter, et al., FEMS Yeast Res. 18:foy009, 2018). While well-studied in the context of ubiquitin-dependent sorting of proteins, none of the aforementioned studies describe the association of MVB12 with alcohol production or tolerance.

Applicants have discovered that yeast having a genetic alteration that reduces MVB12 production demonstrate increased ethanol production in fermentations, and increased butanol tolerance allowing for higher yields, shorter fermentation times, reduced lag phase, or all of these benefits.

The reduction in the amount of functional MVB12 polypeptides can result from disruption of a gene encoding a MVB12 polypeptide (i.e., YGR206W) present in the parental strain. Because disruption of a YGR206W gene is a primary genetic determinant for conferring the altered alcohol production and tolerance phenotype to the modified cells, in some embodiments the modified cells need only comprise a disrupted YGR206W gene, while all other genes can remain intact. In other embodiments, the modified cells can optionally include additional genetic alterations compared to the parental cells from which they are derived. While such additional genetic alterations are not necessary to confer the described phenotype, they may confer other advantages to the modified cells.

Disruption of a YGR206W gene can be performed using any suitable methods that substantially prevent expression of a function MVB12 polypeptides. Exemplary methods of disruption as are known to one of skill in the art include but are not limited to: complete or partial deletion of a YGR206W gene, including complete or partial deletion of, e.g., a MVB12-coding sequence, the promoter, the terminator, an enhancer, or another regulatory element; and complete or partial deletion of a portion of the chromosome that includes any portion of a YGR206W gene. Particular methods of disrupting a YGR206W gene include making nucleotide substitutions or insertions in any portion of a YGR206W gene, e.g., a MVB12-coding sequence, the promoter, the terminator, an enhancer, or another regulatory element. Preferably, deletions, insertions, and/or substitutions (collectively referred to as mutations) are made by genetic manipulation using sequence-specific molecular biology techniques, as opposed to by chemical mutagenesis, which is generally not targeted to specific nucleic acid sequences. Nonetheless, chemical mutagenesis can, in theory, be used to disrupt a YGR206W gene.

Mutations in a YGR206W gene can reduce the efficiency of a YGR206W promoter, reduce the efficiency of a YGR206W enhancer, interfere with the splicing or editing of a YGR206W mRNA, interfere with the translation of a YGR206W mRNA, introduce a stop codon into a MVB12-coding sequence to prevent the translation of full-length MVB12 protein, change the coding sequence of a MVB12 protein to produce a less active or inactive protein or reduce MVB12 interaction with other proteins, or DNA, change the coding sequence of a MVB12 protein to produce a less stable protein or target the protein for destruction, cause a MVB12 protein to misfold or be incorrectly modified (e.g., by glycosylation), or interfere with cellular trafficking of a MVB12 protein. In some embodiments, these and other genetic manipulations act to reduce or prevent the expression of a functional MVB12 protein, or reduce or prevent the normal function of MVB12.

Preferably, disruption of a YGR206W gene is performed by genetic manipulation using sequence-specific molecular biology techniques, as opposed to chemical mutagenesis, which is generally not targeted to specific nucleic acid sequences. However, chemical mutagenesis is not excluded as a method for making modified yeast cells.

In some embodiments, the parental cell that is modified already includes a gene of interest, such as a gene encoding a selectable marker, carbohydrate-processing enzyme, or other polypeptide. In some embodiments, a gene of introduced is subsequently introduced into the modified cells.

It is expected that the present compositions and methods are applicable to other structurally similar MVB12 polypeptides, as well as other related proteins, homologs, and functionally similar polypeptides.

In some embodiments of the present compositions and methods, the amino acid sequence of the MVB12 protein that is altered in production levels has a specified degree of overall amino acid sequence identity to the amino acid sequence of SEQ ID NO: 1, e.g., at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or even at least about 99% identity, to SEQ ID NO: 1.

In some embodiments of the present compositions and methods, the YGR206W gene that is disrupted encodes a MVB12 protein that has a specified degree of overall amino acid sequence identity to the amino acid sequence of SEQ ID NO: 1, e.g., at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or even at least about 99% identity, to SEQ ID NO: 1.

The amino acid sequence information provided, herein, readily allows the skilled person to identify a MVB12 protein, and the nucleic acid sequence encoding a MVB12 protein, in any yeast, and to make appropriate disruptions in a MVB12 gene to affect the production of the MVB12 protein.

In some embodiments, the decrease in the amount of functional MVB12 polypeptide in the modified cells is a decrease of at least 30%, at least 40%, at least 50%, at least 60%, at least 70%, at least 80%, at least 90%, at least 95%, at least 98%, at least 99%, or more, compared to the amount of functional MVB12 polypeptide in parental cells growing under the same conditions. In some embodiments, the reduction of expression of functional MVB12 protein in the modified cells is a reduction of at least 30%, at least 40%, at least 50%, at least 60%, at least 70%, at least 80%, at least 90%, at least 95%, at least 98%, at least 99%, or more, compared to the amount of functional MVB12 polypeptide in parental cells growing under the same conditions.

In some embodiments, the increase in alcohol production by the modified cells is an increase of at least 1%, at least 1.5%, at least 2.0%, at least 2.5%, at least 3%, or more, compared to the amount of alcohol produced in parental cells growing under the same conditions.

In some embodiments, the decrease in acetate production by the modified cells is a decrease of at least 5%, at least 15%, at least 20%, or more, compared to the amount of alcohol produced in parental cells growing under the same conditions.

In some embodiments, the decrease in lag phase of the modified cells in the presence of alcohol is a decrease of at least 10%, at least 20%, at least 30%, at least 40%, or more, compared to the lag phase of parental cells growing under the same conditions.

Combination of Decreased MVB12 Expression with Other Mutations that Affect Alcohol Production

In some embodiments, in addition to expressing decreased amounts of MVB12 polypeptides, the present modified yeast cells further include additional modifications that affect alcohol production.

In particular embodiments the modified yeast cells include an artificial or alternative ethanol-producing pathway resulting from the introduction of a heterologous phosphoketolase (PKL) gene, a heterologous phosphotransacetylase (PTA) gene and a heterologous acetylating acetyl dehydrogenase (AADH), as described in PCT Application Publication No. WO 2015/148272 (Miasnikov, et al.), to channel carbon flux away from the glycerol pathway and toward the synthesis of acetyl-CoA, which is then converted to ethanol.

The modified cells may further include mutations that result in attenuation of the native glycerol biosynthesis pathway, which are known to increase alcohol production. Methods for attenuation of the glycerol biosynthesis pathway in yeast are known and include reduction or elimination of endogenous NAD-dependent glycerol 3-phosphate dehydrogenase (GPD) or glycerol phosphate phosphatase activity (GPP), for example by disruption of one or more of the genes GPD1, GPD2, GPP1 and/or GPP2. See, e.g., U.S. Pat. No. 9,175,270 (Elke, et al.), U.S. Pat. No. 8,795,998 (Pronk, et al.), and U.S. Pat. No. 8,956,851 (Argyros, et al.).

The modified yeast may further feature increased acetyl-CoA synthase (also referred to acetyl-CoA ligase) activity (Enzyme Commission Number 6.2.1.1) to scavenge (i.e., capture) acetate produced by chemical or enzymatic hydrolysis of acetyl-phosphate (or present in the culture medium of the yeast for any other reason) and converts it to Ac-CoA. This avoids the undesirable effect of acetate on the growth of yeast cells and may further contribute to an improvement in alcohol yield. Increasing acetyl-CoA synthase activity may be accomplished by introducing a heterologous acetyl-CoA synthase gene into cells, increasing the expression of an endogenous acetyl-CoA synthase gene and the like. A particularly useful acetyl-CoA synthase for introduction into cells can be obtained from Methanosaeta concilii (UniProt/TrEMBL Accession No.: WP_013718460). Homologs of this enzymes, including enzymes having at least 85%, at least 90%, at least 92%, at least 95%, at least 97%, at least 98% and even at least 99% amino acid sequence identity to the aforementioned acetyl-CoA synthase from Methanosaeta concilii, are also useful in the present compositions and methods.

In some embodiments, the modified cells may further include a heterologous gene encoding a protein with NAD⁺-dependent acetylating acetaldehyde dehydrogenase activity and/or a heterologous gene encoding a pyruvate-formate lyase. The introduction of such genes in combination with attenuation of the glycerol pathway is described, e.g., in U.S. Pat. No. 8,795,998 (Pronk, et al.).

In some embodiments, the present modified yeast cells may further overexpress a sugar transporter-like (STL1) polypeptide (see, e.g., Ferreira, et al., Mol Biol Cell 16:2068-76, 2005; Duskovi, et al., Mol Microbiol 97:541-59, 2015, and PCT Application Publication No. WO 2015/023989) to increase ethanol production and reduce acetate.

In some embodiments, the present modified yeast cells may further overexpress a polyadenylate-binding protein, e.g., PAB1, to increase alcohol production and reduce acetate production.

In some embodiments, the present modified yeast cells further comprise a butanol biosynthetic pathway. In some embodiments, the butanol biosynthetic pathway is an isobutanol biosynthetic pathway. In some embodiments, the isobutanol biosynthetic pathway comprises a polynucleotide encoding a polypeptide that catalyzes a substrate to product conversion selected from the group consisting of: (a) pyruvate to acetolactate; (b) acetolactate to 2,3-dihydroxyisovalerate; (c) 2,3-dihydroxyisovalerate to α-ketoisovalerate; (d) α-ketoisovalerate to isobutyraldehyde; and (e) isobutyraldehyde to isobutanol. In some embodiments, the isobutanol biosynthetic pathway comprises polynucleotides encoding polypeptides having acetolactate synthase, ketol-acid reductoisomerase, dihydroxy acid dehydratase, branched-chain α-keto acid decarboxylase, and alcohol dehydrogenase activities.

Genes and polypeptides that can be used for substrate to product conversions are described herein and/or in the art, for example, in U.S. Pat. No. 7,851,188. Ketol-acid reductoisomerase enzymes are described in U.S. Patent Application Publication Nos. 2008/0261230, 2009/0163376, 2010/0197519, and PCT Application Publication No. WO 2011/1041415. Examples of KARIs disclosed therein are those from Lactococcus lactis, Vibrio cholera, Pseudomonas aeruginosa PAO1, as well as Pseudomonasfluorescens PF5 mutants. KARIs include Anaerostipes caccae KARI as well as variants are described in U.S. Pat. Nos. 10,174,345; 9,512,408; 9,422,581; 9,422,582; and 9,790,521. Each of the above-referenced applications and patents is herein incorporated by reference.

U.S. Patent Application Publication No. 2010/0081154 and U.S. Pat. No. 7,851,188 describe dihydroxyacid dehydratases (DHADs), including a DHAD from Streptococcus mutans (see, also, U.S. Pat. No. 9,580,705, herein incorporated by reference). Suitable polypeptides to catalyze the conversion of a-ketoisovalerate to isobutyraldehyde include those from Listeria grayi, Lactococcus lactis, and Macrococcus caseolyticus described in U.S. Pat. No. 9,169,467. U.S. Patent Application Publication No. 2009/0269823 describes SadB, an alcohol dehydrogenase (ADH) from Achromobacter xylosoxidans. Alcohol dehydrogenases also include horse liver ADH and Beijerinkia indica ADH described in U.S. Pat. No. 8,765,433. Each of the above-referenced applications and patents is herein incorporated by reference.

In some embodiments, the isobutanol biosynthetic pathway comprises at least one gene, at least two genes, at least three genes, or at least four genes that is/are heterologous to the modified yeast cell. In some embodiments, the modified yeast cell comprises a heterologous gene for each substrate to product conversion of an isobutanol biosynthetic pathway. In embodiments, the polypeptide catalyzing the substrate to product conversions of acetolactate to 2,3-dihydroxyisovalerate and/or the polypeptide catalyzing the substrate to product conversion of isobutyraldehyde to isobutanol are capable of utilizing NADH as a cofactor.

In other embodiments, the modified yeast cells can have reduced or substantially eliminated expression of a polypeptide which catalyzes the conversion of glycerol-3-phosphate into dihydroxyacetone phosphate. In embodiments, the polypeptide which catalyzes the conversion of glycerol-3-phosphate into dihydroxyacetone phosphate is glycerol-3-phosphate dehydrogenase (GPD). In embodiments, the modified yeast cell comprises a deletion, mutation, and/or substitution in an endogenous polynucleotide encoding a polypeptide which catalyzes the conversion of glycerol-3-phosphate into dihydroxyacetone phosphate. In embodiments, the polypeptide which catalyzes the conversion of glycerol-3-phosphate into dihydroxyacetone phosphate corresponds to Enzyme Commission Number 1.1.1.8. In embodiments, the polynucleotide encoding a polypeptide which catalyzes the conversion of glycerol-3-phosphate into dihydroxyacetone phosphate is GPD1 or GPD2. Such modifications and others to modified yeast cells are described in U.S. Patent Application Publication No. 2009/0305363, the entire contents of which is herein incorporated by reference.

Endogenous pyruvate decarboxylase activity in microbial cells converts pyruvate to acetaldehyde, which is then converted to ethanol or to acetyl-CoA via acetate. Microbial cells can have one or more genes encoding pyruvate decarboxylase. For example, in yeast, there is one gene encoding pyruvate decarboxylase in Kluyveromyces lactis, while there are three isozymes of pyruvate decarboxylase encoded by the PDC1, PDC5, and PDC6 genes in Saccharomyces cerevisiae, as well as a pyruvate decarboxylase regulatory gene PDC2. Expression of pyruvate decarboxylase from PDC6 is minimal. In embodiments of the invention, the yeast cells can have pyruvate decarboxylase activity that is reduced by disrupting at least one gene encoding a pyruvate decarboxylase, or a gene regulating pyruvate decarboxylase gene expression. For example, the PDC1 and PDC5 genes, or all three genes, are disrupted. In addition, pyruvate decarboxylase activity can be reduced by disrupting the PDC2 regulatory gene in the yeast cells. Polypeptides having PDC activity or a polynucleotide or gene encoding a polypeptide having PDC activity corresponds to Enzyme Commission Number EC 4.1.1.1. Such modifications and others to modified yeast cells are described in U.S. Pat. No. 9,790,521, the entire contents of which is herein incorporated by reference.

In embodiments, the yeast cells of the invention can have expression of pyruvate decarboxylase and/or glycerol-3-phosphate dehydrogenase that is decreased or substantially eliminated. In other embodiments, the yeast cells comprise a deletion, mutation, and/or substitution in an endogenous polynucleotide encoding a polypeptide having the activity of pyruvate decarboxylase or glycerol-3-phosphate dehydrogenase.

Other modifications include at least one deletion, mutation, and/or substitution in an endogenous polynucleotide encoding a polypeptide having acetolactate reductase activity. In embodiments, the polypeptide having acetolactate reductase activity is YMR226C of Saccharomyces cerevisiae or a homolog thereof. Additional modifications include a deletion, mutation, and/or substitution in an endogenous polynucleotide encoding a polypeptide having aldehyde dehydrogenase and/or aldehyde oxidase activity. Such modifications and others to modified yeast cells are described in U.S. Pat. No. 9,790,521, the entire contents of which is herein incorporated by reference. In embodiments, the polypeptide having aldehyde dehydrogenase activity is ALD6 from Saccharomyces cerevisiae or a homolog thereof. In some embodiments, the yeast cells further comprise a deletion, mutation, and/or substitution in one or more endogenous polynucleotides encoding FRA2, ADH1, and BDH1. Such modifications and others to modified yeast cells are described in U.S. Pat. No. 9,297,016, the entire contents of which is herein incorporated by reference.

In some embodiments, the yeast cells of the invention have reduced expression of MVB12 and an isobutanol biosynthetic pathway. In another embodiment, the yeast cells of the invention with reduced expression of MVB12 can be used in a co-fermentation process with another yeast engineered to produce isobutanol, so that ethanol and isobutanol are both produced in a single fermentation vessel.

Combination of Decreased MVB12 Expression with Other Beneficial Mutations

In some embodiments, in addition to expressing reduced amounts of MVB12 polypeptides, optionally in combination with other genetic modifications that benefit alcohol production, the present modified yeast cells further include any number of additional genes of interest encoding proteins of interest. Additional genes of interest may be introduced before, during, or after genetic manipulations that result in reduced expression of MVB12. Proteins of interest, include selectable markers, carbohydrate-processing enzymes, and other commercially-relevant polypeptides, including but not limited to an enzyme selected from the group consisting of a dehydrogenase, a transketolase, a phosphoketolase, a transladolase, an epimerase, a phytase, a xylanase, a β-glucanase, a phosphatase, a protease, an α-amylase, a β-amylase, a glucoamylase, a pullulanase, an isoamylase, a cellulase, a trehalase, a lipase, a pectinase, a polyesterase, a cutinase, an oxidase, a transferase, a reductase, a hemicellulase, a mannanase, an esterase, an isomerase, a pectinases, a lactase, a peroxidase and a laccase. Proteins of interest may be secreted, glycosylated, and otherwise-modified.

### Yeast Cells Suitable for Modification

Yeasts are unicellular eukaryotic microorganisms classified as members of the fungus kingdom and include organisms from the phyla Ascomycota and Basidiomycota. Yeast that can be used for alcohol production include, but are not limited to, Saccharomyces spp., including Saccharomyces cerevisiae, as well as Kluyveromyces, Lachancea, Zygosaccharomyces, Candida, and Schizosaccharomyces spp. Species of yeast include, but are not limited to, Saccharomyces kluyveri, Schizosaccharomyces pombe, Saccharomyces bayanus, Saccharomyces mikitae, Saccharomyces paradoxus, Zygosaccharomyces rouxii, and Candida glabrata. Numerous yeast strains are commercially available, many of which have been selected or genetically engineered for desired characteristics, such as high alcohol production, rapid growth rate, and the like. Some yeasts have been genetically engineered to produce heterologous enzymes, such as glucoamylase or α-amylase.

### Substrates and Products

Alcohol production from a number of carbohydrate substrates, including but not limited to corn starch, sugar cane, cassava, and molasses, is well known, as are innumerable variations and improvements to enzymatic and chemical conditions and mechanical processes. The present compositions and methods are believed to be fully compatible with such substrates and conditions.

### Industrial Batch and Continuous Fermentations

Isobutanol, or other products, may be produced using a batch method of fermentation. A classical batch fermentation is a closed system where the composition of the medium is set at the beginning of the fermentation and not subject to artificial alterations during the fermentation. A variation on the standard batch system is the fed-batch system. Fed-batch fermentation processes are also suitable in the present invention and comprise a typical batch system with the exception that the substrate is added in increments as the fermentation progresses. Fed-batch systems are useful when catabolite repression is apt to inhibit the metabolism of the cells and where it is desirable to have limited amounts of substrate in the media. Batch and fed-batch fermentations are common and well known in the art and examples may be found in Thomas D. Brock in Biotechnology: A Textbook of Industrial Microbiology, Second Edition (1989) Sinauer Associates, Inc., Sunderland, Mass., or Deshpande, Appl. Biochem. Biotechnol. 36:227, 1992.

Isobutanol, or other products, may also be produced using continuous fermentation methods. Continuous fermentation is an open system where a defined fermentation medium is added continuously to a bioreactor and an equal amount of conditioned media is removed simultaneously for processing. Continuous fermentation generally maintains the cultures at a constant high density where cells are primarily in log phase growth. Continuous fermentation allows for the modulation of one factor or any number of factors that affect cell growth or end product concentration. Methods of modulating nutrients and growth factors for continuous fermentation processes as well as techniques for maximizing the rate of product formation are well known in the art of industrial microbiology and a variety of methods are detailed by Brock, supra.

It is contemplated that the production of isobutanol, or other products, may be practiced using batch, fed-batch, or continuous processes and that any known mode of fermentation would be suitable. Additionally, it is contemplated that cells may be immobilized on a substrate as whole cell catalysts and subjected to fermentation conditions for isobutanol production.

Alcohol fermentation products include organic compound having a hydroxyl functional group (—OH) is bound to a carbon atom. Exemplary alcohols include but are not limited to methanol, ethanol, n-propanol, isopropanol, n-butanol, isobutanol, n-pentanol, 2-pentanol, isopentanol, and higher alcohols. The most commonly made fuel alcohols are ethanol, and butanol.

Methods for Isobutanol Isolation from the Fermentation Medium

Bioproduced isobutanol may be isolated from the fermentation medium using methods known in the art, for example, ABE fermentations (see, e.g., Durre, Appl. Microbiol. Biotechnol. 49:639-648, 1998, Groot, et al., Process. Biochem. 27:61-75, 1992, and references therein). For example, solids may be removed from the fermentation medium by centrifugation, filtration, decantation, or the like (see, e.g., U.S. Patent Application Publication No. 2012/0164302, the entire contents of which are herein incorporated by reference). Isobutanol may be isolated from the fermentation medium using methods such as distillation, azeotropic distillation, liquid-liquid extraction, adsorption, gas stripping, membrane evaporation, or pervaporation.

Isobutanol may also be isolated from the fermentation medium using liquid-liquid extraction in combination with distillation. In this method, isobutanol is extracted from the fermentation broth using liquid-liquid extraction with a suitable solvent. The isobutanol-containing organic phase is then distilled to separate isobutanol from the solvent. Additionally, isobutanol may be isolated using azeotropic distillation using an entrainer (see, e.g., Doherty and Malone, Conceptual Design of Distillation Systems, McGraw Hill, New York, 2001).

Distillation in combination with adsorption may also be used to isolate isobutanol from the fermentation medium. In this method, the fermentation broth containing isobutanol is distilled to near the azeotropic composition and then the remaining water is removed by use of an adsorbent, such as molecular sieves (Aden, et al., Lignocellulosic Biomass to Ethanol Process Design and Economics Utilizing Co-Current Dilute Acid Prehydrolysis and Enzymatic Hydrolysis for Corn Stover, Report NREL/TP-510-32438, National Renewable Energy Laboratory, June 2002).

Additionally, distillation in combination with pervaporation may be used to isolate and purify isobutanol from the fermentation medium. In this method, the fermentation broth containing isobutanol is distilled to near the azeotropic composition, and then the remaining water is removed by pervaporation through a hydrophilic membrane (Guo, et al., J. Membr. Sci. 245:199-210, 2004).

In situ product removal (ISPR) (also referred to as extractive fermentation) may be used to remove isobutanol (or other fermentative alcohol) from the fermentation vessel as it is produced, thereby allowing the yeast to produce isobutanol at high yields. One method for ISPR for removing fermentative alcohol that has been described in the art is liquid-liquid extraction. In general, with regard to isobutanol fermentation, for example, the fermentation medium, which includes the yeast, is contacted with an organic extractant at a time before the isobutanol concentration reaches a toxic level. The organic extractant and the fermentation medium form a biphasic mixture. Isobutanol partitions into the organic extractant phase, decreasing the concentration in the aqueous phase containing the yeast, thereby limiting the exposure of the yeast to the inhibitory isobutanol. Liquid-liquid extraction may be performed, for example, according to the processes described in U.S. Patent Application Publication No. 2009/0305370; U.S. Patent Application Publication No. 2011/0097773; U.S. Patent Application Publication No. 2012/0156738, the disclosures of which is hereby incorporated in its entirety.

These and other aspects and embodiments of the present strains and methods will be apparent to the skilled person in view of the present description. The following examples are intended to further illustrate, but not limit, the strains and methods.

### EXAMPLES

### Example 1: Reduced Expression of MVB12 in S. cerevisiae

Using standard yeast molecular biology techniques, the YGR206W gene was disrupted by deleting essentially the entire coding sequence for MVB12. All procedures were based on the publicly available nucleic acid sequence of YGR206W (chrVII:910432 . . . 910737), which is provided below (5′ to 3′), as SEQ ID NO: 2:

ATGAACAATAATGTTGAAGAACTCCTACGGCGTATTCCCCTCTACAACAA
ATATGGTAAGGATTTTCCACAAGAAACAGTGACTAGGTTTCAGATGCCTG
AATTTAAGTTACCAGCGCTACAACCAACAAGGGACTTATTATGCCCTTGG
TACGAAGAATGTGACAATATCACCAAAGTCTGTCAGTTACATGATTCTTC
TAATAAGAAATTCGATCAGTGGTACAAGGAGCAATATCTGAGTAAAAAAC
CACCGGGAATTGTTGGAAACACACTATTATCTCCATCAAGAAAGGATAAT
TCCTAG

The host yeast used to make the modified yeast cells was commercially available FERMAX™ Gold (Martrex, Inc., Chaska, Minn., USA, herein “FG”). Deletion of the YGR206W gene was confirmed by colony PCR. The modified yeast was grown in non-selective media to remove the plasmid conferring Kanamycin resistance used to select transformants, resulting in modified yeast that required no growth supplements compared to the parental yeast. One modified strain, designated FG-mvb12, was selected for further study.

### Example 2: Ethanol Production by Yeast with Reduced Expression of MVB12

FG-mvb12 yeast harboring the deletion of the YGR206W gene was tested for its ability to produce ethanol compared to the FG benchmark yeast, which is wild-type for the YGR206W gene, in liquefact incubated at in a 32° C. or in a 32-35° C. ramp. The liquefact was corn flour slurry having a dry solid (ds) value of 35%, prepared by combining 600 ppm urea, 0.124 SAPU/g ds FERMGEN™ 2.5× (an acid fungal protease), 0.33 GAU/g ds variant Trichoderma reesei glucoamylase and 1.46 SSCU/g ds Aspergillus kawachii α-amylase, at pH 4.8.

Liquefact (50 grams) was weighted into 100 ml vessels and inoculated with fresh overnight cultures from colonies of the modified strain or FG strain at 32° C. or in the 32-35° C. ramp. Samples were harvested by centrifugation at 24, 48, 55 and 72 hr following inoculation, filtered through 0.2 μm filters, and analyzed for ethanol, glucose, acetate and glycerol content by IPLC (Agilent Technologies 1200 series) using Bio-Rad Aminex HPX-87H columns at 55° C. with an isocratic flow rate of 0.6 ml/min in 0.01 N H₂SO₄ eluent. A 2.5 μl sample injection volume was used. Calibration standards used for quantification included known amounts of DP4+, DP3, DP2, DP1, glycerol and ethanol. The results of the analyses are shown in Table 1. Ethanol increase is reported with reference to the FG strain.

TABLE 1
Analysis of fermentation broth following
fermentation under described conditions

| | | | | | Ethanol |
|---|---|---|---|---|---|
| | Temp. | Time | Glucose | Ethanol | Increase |
| Strain | (° C.) | (hr) | (g/L) | (g/L) | (%) |
| FG | 32 | 48 | 4.92 | 140.95 | -0- |
| FG-mvb12 | 32 | 48 | 0.943 | 143.52 | 1.82 |
| FG | 32 | 55 | 1.90 | 142.62 | -0- |
| FG-mvb12 | 32 | 55 | 0.319 | 143.92 | 0.92 |
| FG | 32-35 ramp | 48 | 15.83 | 137.49 | -0- |
| FG-mvb12 | 32-35 ramp | 48 | 7.13 | 140.90 | 2.48 |
| FG | 32-35 ramp | 55 | 10.95 | 139.54 | -0- |
| FG-mvb12 | 32-35 ramp | 55 | 4.75 | 141.17 | 1.17 |

Yeast harboring the YGR206W gene deletion produced up to about 2.5% more ethanol compared the unmodified reference strain at 32° C. and under the 32-35° C. ramp conditions.

### Example 3: Acetate Production by Modified Yeast with Reduced Expression of MVB12

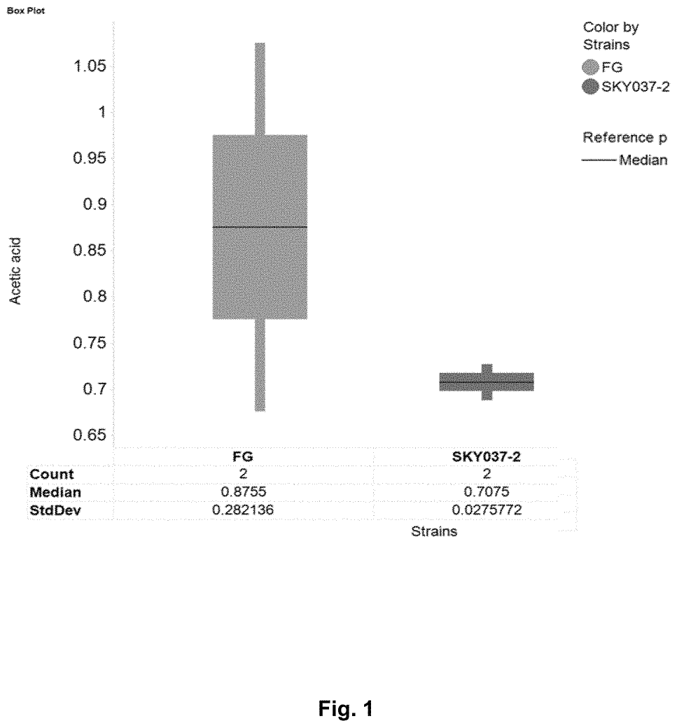

Using similar methods as described in Example 2, acetate production was measured in FG-mvb12 yeast harboring the deletion of the YGR206W compared to the parental yeast. FG-mvb12 yeast had a lower acetate production (i.e., about 20%) after 55 hours of fermentation at 32° C. (FIG. 1).

### Example 4: Increased Isobutanol Tolerance by Yeast with Reduced Expression of MVB12

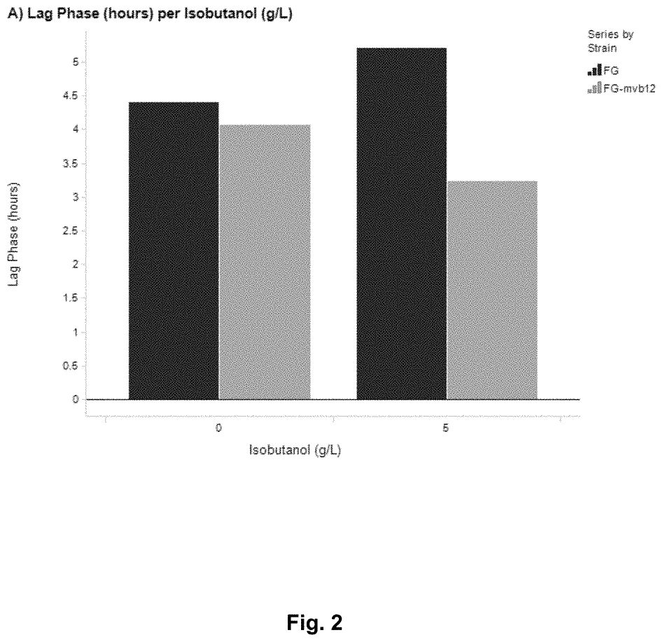

To test for increased tolerance to isobutanol the strains were grown in clarified liquefact consisting of corn flour slurry having a dry solid (ds) value of 35% was filtered through a 0.2 μm filter to remove all the particulates and supplemented with 50 g/L glucose and 600 ppm urea with 5 g/L isobutanol for 65 hours to assess the lag phase, maximum exponential growth and stationary phase performance by fitting a Gompertz model (Zwietering, et al., Appl. Environ. Microbiol. 56:1875-81, 1990). As shown in the graph in FIG. 2, the strain harboring the MVB12 deletion had a significantly shorter growth lag phase (i.e., about 40%) in the presences of 5 g/L isobutanol.

### Example 5: Reduced Expression of MVB12 in an Isobutanologen Strain

A yeast cell comprising an isobutanol pathway was constructed as described in Example 1 of U.S. Pat. No. 10,280,438 (identified as strain PNY1621). A deletion of YGR206W is constructed using CRISPR by transformation of PNY1621 with Cas9-gRNA plasmid and deletion cassette using standard techniques described in Generoso, et al. (J. Microbiological Methods 127:203-205, 2016). An example gRNA sequence for YGR206W is 5′-TGTCACATTCTTCGTACCAA (SEQ ID NO: 3) with PAM sequence GGG. Example oligonucleotides for an annealed deletion cassette are 5′-GATTAAAAAGGAAAGGAAAATAGCAATGGGAGCTTATCGCATAAAAAATTAAATTG CATTTCGATATCTATGTACACATATACAGCAATTTTTTTTTATAAACC (SEQ ID NO: 4) and 5′-GGTTTATAAAAAAAAATTGCTGTATATGTGTACATAGATATCGAAATGCA ATTTAATTTTTTATGCGATAAGCTCCCATTGCTATTTTCCTTTCCTTTTTAATC(SEQ ID NO: 5). The resulting strain is checked by colony PCR to confirm deletion of the YGR206W sequence and is called PNY1621 mvb211.

While various embodiments of the present invention have been described above, it should be understood that they have been presented by way of example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the following claims and their equivalents.

All publications, patents and patent applications mentioned in this specification are indicative of the level of skill of those skilled in the art to which this invention pertains, and are herein incorporated by reference to the same extent as if each individual publication, patent or patent application was specifically and individually indicated to be incorporated by reference.

What is claimed is:

1. Modified yeast cells derived from parental yeast cells, the modified cells comprising a genetic alteration that causes the modified cells to produce a decreased amount of functional MVB12 polypeptides compared to the parental cells, wherein the modified cells have increased alcohol tolerance and/or decreased alcohol tolerance compared to the parental cells under equivalent fermentation conditions.

2. The modified cells of claim 1, wherein the genetic alteration comprises a disruption of a YGR206W gene present in the parental cells.

3. The modified cells of claim 2, wherein disruption of a YGR206W gene is the result of deletion of all or part of a YGR206W gene.

4. The modified cells of claim 2, wherein disruption of a YGR206W gene is the result of deletion of a portion of genomic DNA comprising a YGR206W gene.

5. The modified cells of claim 2, wherein disruption of a YGR206W gene is the result of mutagenesis of a YGR206W gene.

6. The modified cells of claim 2, wherein disruption of a YGR206W gene is performed in combination with introducing a gene of interest at the genetic locus of a YGR206W gene.

7. The modified cells of claim 1, wherein the cells do not produce functional MVB12 polypeptides.

8. The modified cells of claim 1, wherein the cells do not produce MVB12 polypeptides.

9. The modified cells of claim 1, wherein the cells further comprise an exogenous gene encoding a carbohydrate processing enzyme.

10. The modified cells of claim 1, further comprising an alteration in the glycerol pathway and/or the acetyl-CoA pathway.

11. The modified cells of claim 1, further comprising an alternative pathway for making alcohol.

12. The modified cells of claim 1, wherein the cells are of a Saccharomyces spp.

13. The modified cells of claim 1, wherein the cells produce an increased amount of ethanol compared to the parental cells.

14. The modified cells of claim 1, wherein the cells produce a decreased amount of acetate compared to the parental cells.

15. The modified cells of claim 1, wherein the cells have a reduced lag phase in the presence of butanol compared to the parental cells.

16. The modified cells of claim 1, further comprising an isobutanol biosynthetic pathway.

17. The modified cells of claim 16, wherein the isobutanol biosynthetic pathway comprises a polynucleotide encoding a polypeptide that catalyzes a substrate to product conversion selected from the group consisting of: (a) pyruvate to acetolactate; (b) acetolactate to 2,3-dihydroxyisovalerate; (c) 2,3-dihydroxyisovalerate to α-ketoisovalerate; (d) α-ketoisovalerate to isobutyraldehyde; and (e) isobutyraldehyde to isobutanol.

18. A method for producing a modified yeast cell comprising: introducing a genetic alteration into a parental yeast cell, which genetic alteration reduces or prevents the production of functional MVB12 polypeptide compared to the parental cells, thereby producing modified cells that have during fermentation an increased alcohol tolerance and/or decreased alcohol tolerance compared to the parental cells under equivalent fermentation.

19. The method of claim 18, wherein the genetic alteration comprises disrupting a YGR206W gene in the parental cells by genetic manipulation.

20. The method of claim 18, wherein the genetic alteration comprises deleting a YGR206W gene in the parental cells using genetic manipulation.

21. The method of claim 18, wherein disruption of a YGR206W gene is performed in combination with introducing a gene of interest at the genetic locus of a YGR206W gene.

22. The method of claim 18, wherein disruption of a YGR206W gene is performed in combination with making an alteration in the glycerol pathway and/or the acetyl-CoA pathway.

23. The method of claim 18, wherein disruption of a YGR206W gene is performed in combination with adding an alternative pathway for making alcohol.

24. The method of claim 18, wherein disruption of a YGR206W gene is performed in combination with introducing an exogenous gene encoding a carbohydrate processing enzyme.

25. The method of claim 18, wherein the modified yeast cell further comprises an isobutanol biosynthetic pathway.

26. The modified cells of claim 25, wherein the isobutanol biosynthetic pathway comprises a polynucleotide encoding a polypeptide that catalyzes a substrate to product conversion selected from the group consisting of: (a) pyruvate to acetolactate; (b) acetolactate to 2,3-dihydroxyisovalerate; (c) 2,3-dihydroxyisovalerate to α-ketoisovalerate; (d) α-ketoisovalerate to isobutyraldehyde; and (e) isobutyraldehyde to isobutanol.

27. The method of claim 18, wherein the modified cell is from a Saccharomyces spp.

28. Modified yeast cells produced by the method of claim 18.


SEQUENCE LISTING (5 sequences)

SEQ ID NO: 1 - protein, 101 aa, Saccharomyces cerevisiae
MNNNVEELLRRIPLYNKYGKDFPQETVTRFQMPEFKLPALQPTRDLLCPWYEECDNITKVCQLHDSSNKKFDQWYKEQYLSKKPPGIVGNTLLSPSRKDNS

SEQ ID NO: 2 - DNA, 306 bp, Saccharomyces cerevisiae
atgaacaataatgttgaagaactcctacggcgtattcccctctacaacaaatatggtaaggattttccacaagaaacagtgactaggtttcagatgcctgaatttaagttaccagcgctacaaccaacaagggacttattatgcccttggtacgaagaatgtgacaatatcaccaaagtctgtcagttacatgattcttctaataagaaattcgatcagtggtacaaggagcaatatctgagtaaaaaaccaccgggaattgttggaaacacactattatctccatcaagaaaggataattcctag

SEQ ID NO: 3 - DNA, 20 bp, Artificial Sequence
  gRNA sequence for YGR206W
tgtcacattcttcgtaccaa

SEQ ID NO: 4 - DNA, 104 bp, Artificial Sequence
  Oligonucleotide
gattaaaaaggaaaggaaaatagcaatgggagcttatcgcataaaaaattaaattgcatttcgatatctatgtacacatatacagcaatttttttttataaacc

SEQ ID NO: 5 - DNA, 104 bp, Artificial Sequence
  Oligonucleotide
ggtttataaaaaaaaattgctgtatatgtgtacatagatatcgaaatgcaatttaattttttatgcgataagctcccattgctattttcctttcctttttaatc